(19) United States
(12) Patent Application Publication
(10) Pub. No.: US 2004/0097791 A1
(43) Pub. Date: May 20, 2004

(54) Endoscope

(75) Inventors: Kazunari Tokuda, Tokyo (JP); Tadashi Hirata, Tokyo (JP); Yoshiyuki Kumada, Tokyo (JP)

Correspondence Address: SCULLY SCOTT MURPHY & PRESSER, PC, 400 GARDEN CITY PLAZA, GARDEN CITY, NY, 11530

(73) Assignee: Olympus Corporation, Tokyo (JP)

(21) Appl. No.: 10/706,590

(22) Filed: Nov. 12, 2003

(30) Foreign Application Priority Data
Nov. 13, 2002 (JP) 2002-329898
Nov. 13, 2002 (JP) 2002-329869

(51) Int. Cl. A61B 1/06

(52) U.S. Cl. 600/173; 600/167; 600/168

(57) ABSTRACT

An endoscope includes an optical system for endoscope observation arranged in an edge of an inserting portion which can be inserted in a specimen, an optical system for microscopic observation which microscopically observes an observing portion of the specimen, a moving mechanism for adjusting an angle of view which changes the angle of view by moving a part of the optical system for endoscope observation, and a focal-point adjusting mechanism which is arranged in the edge of the inserting portion and which moves a focal point on the side of a subject in the optical system for microscopic observation, wherein the moving mechanism for adjusting the angle of view and the focal-point adjusting mechanism are arranged forward and backward of the same axis to the inserting portion.

This application claims benefits of Japanese Applications No. 2002-329868 filed in Japan on Nov. 13, 2002, and No. 2002-329869 filed in Japan on Nov. 13, 2002, the contents of which are incorporated by this reference.

### BACKGROUND OF THE INVENTION

1. Field of the Invention

The present invention relates to an endoscope which is capable of obtaining a normal observing image and an enlarged observing image.

2. Description of the Related Art

Recently, in the case of diagnosis of the organ, various observing systems are proposed. For example, the observing systems include one using an OCT (Optical Coherence Tomography) for obtaining a tomographic image of a specimen by using low-coherent light and one using a confocal image.

In general, the observing systems use the OCT or the confocal image together with normal endoscopic observation. The present applicant proposed, in Japanese Unexamined Patent Application Publication No. 2000-131221, a technology by which an optical scanning probe is inserted into a treatment tool inserting channel arranged to an endoscope and the observation is performed by both the endoscope and the optical scanning probe.

Referring to FIG. 25, as disclosed in Japanese Unexamined Patent Application Publication No. 2000-131221, a system 161 comprises: an endoscope 162 which incorporates an image pick-up device for obtaining an observing image at a wavelength area of visible light; a confocal optical scanning probe 163 which can be used by being inserted in a treatment tool inserting channel arranged to the endoscope 162 and which two-dimensionally scans light with a confocal relationship; an optical tomographic image probe 164 which obtains information on an optical tomography by low-coherent light; and an optical scanning/endoscope control device 165 which detachably connects the endoscope 162, the confocal optical scanning probe 163, and the optical tomographic image probe 164.

Upon observing, by using the system 161, an abnormal portion, e.g., a concerned portion such as a lesion portion in the medical field, first, a connector 167a fixed to the edge of a universal cable 167 extending from an operating portion 166 in the endoscope 162 is connected to an endoscope socket 165a arranged to the optical scanning/endoscope control device 165 and then the normal endoscope observation is performed.

When the abnormal portion is found, the best optical scanning probe is selected for observing the portion and then the portion is further specifically observed. For example, when the confocal optical scanning probe 163 is selected as the optical scanning probe, the confocal optical scanning probe 163 is inserted into the treatment tool inserting channel arranged to the endoscope 162 and the connector 163a in the donfocal optical scanning probe 163 is connected to a socket 165b for probe connection arranged to the optical scanning/endoscope control device 165. The fine structure of the concerned portion is enlarged with a confocal image and is observed by using the confocal optical scanning probe 163.

In addition to the normal observation, the endoscope further obtains the optical tomography by using the low-coherent light for the purpose of the detailed diagnosis of the concerned portion such as the lesion portion. The Japanese Patent No. 3325056 (Japanese Unexamined Patent Application Publication No. 6-154228) discloses an endoscope which can obtain both the normal endoscope image and the optical tomography using the low-coherent light, by using the single endoscope.

Further, in an endoscope as disclosed in Japanese Patent No. 3325056 (Japanese Unexamined Patent Application Publication No. 6-154228), an objective optical system is shared for the normal observation and the optical tomography. Therefore, the edge of an inserting portion in the endoscope can have a thin diameter.

### SUMMARY OF THE INVENTION

According to the present invention, an endoscope includes an optical system for endoscope observation, an optical system for microscopic observation which microscopically observes an observing portion of a specimen, a moving mechanism for adjusting an angle of view which changes the angle of view by moving a part of the optical system for endoscope observation, and a focal-point adjusting mechanism which moves a focal point on the side of a subject in the optical system for microscopic observation, those are arranged in an edge of an inserting portion which can be inserted in the specimen, wherein the moving mechanism for adjusting the angle of view and the focal-point adjusting mechanism are arranged forward and backward of the same axis to the inserting portion.

The above and other objects, features, and advantages of the invention will become more clearly understood from the following description referring to the accompanying drawings.

### BRIEF DESCRIPTION OF THE DRAWINGS

FIGS. 1 to 4 show a first embodiment of the present invention,

FIG. 1 is a block diagram showing the system structure of an endoscope apparatus;

FIG. 2 is a front view showing an edge portion of an endoscope;

FIG. 3 is a cross-sectional view showing the edge portion of the endoscope; and

FIG. 4 is an enlarged cross-sectional view showing a linear actuator;

FIGS. 5 to 7 show a second embodiment of the present invention,

FIG. 5 is a cross-sectional view showing an edge portion of an endoscope and V-V cross-sectional view of FIG. 6;

FIG. 6 is a front view showing one example of the edge portion of the endoscope;

FIG. 7 is a front view showing another example of the edge portion of the endoscope corresponding to FIG. 6;

FIG. 8 is a cross-sectional view showing an edge portion of an endoscope according to a third embodiment of the present invention, corresponding to FIG. 3;

FIG. 9 is a cross-sectional view showing an edge portion of an endoscope according to a fourth embodiment of the present invention, corresponding to FIG. 3;

FIGS. 10 to 14 show a fifth embodiment of the present invention,

FIG. 10 is a diagram showing the system structure of an endoscope apparatus;

FIG. 11 is a cross-sectional view showing an edge portion of an endoscope upon normal observation using the endoscope;

FIG. 12 is a cross-sectional view showing the edge portion of the endoscope upon microscopic observation;

FIG. 13 is an explanatory diagram showing an observing image upon the normal observation using the endoscope;

FIG. 14 is an explanatory diagram showing an observing image upon the microscopic observation.

FIGS. 15 to 17 show a sixth embodiment of the present invention,

FIG. 15 is a diagram showing the system structure of an endoscope apparatus;

FIG. 16 is a cross-sectional view showing an edge portion of an endoscope;

FIG. 17 is a front view showing a scanning mirror;

FIG. 18 is a cross-sectional view showing an edge portion of an endoscope according to a seventh embodiment of the present invention;

FIG. 19 is a cross-sectional view showing an edge portion of an endoscope according to an eighth embodiment of the present invention;

FIG. 20 is a cross-sectional view showing an edge portion of an endoscope according to a ninth embodiment of the present invention;

FIGS. 21 and 22 show a tenth embodiment of the present invention,

FIG. 21 is a cross-sectional view showing an edge portion of an endoscope;

FIG. 22 is a perspective view showing a piezoelectric actuator;

FIGS. 23 and 24 show an eleventh embodiment of the present invention,

FIG. 23 is a diagram showing the system structure of an endoscope apparatus;

FIG. 24 is a cross-sectional view showing an edge portion of an endoscope; and

FIG. 25 is a block diagram schematically showing the structure of a conventional endoscope unit.

### DESCRIPTION OF THE PREFERRED EMBODIMENTS

(First Embodiment)

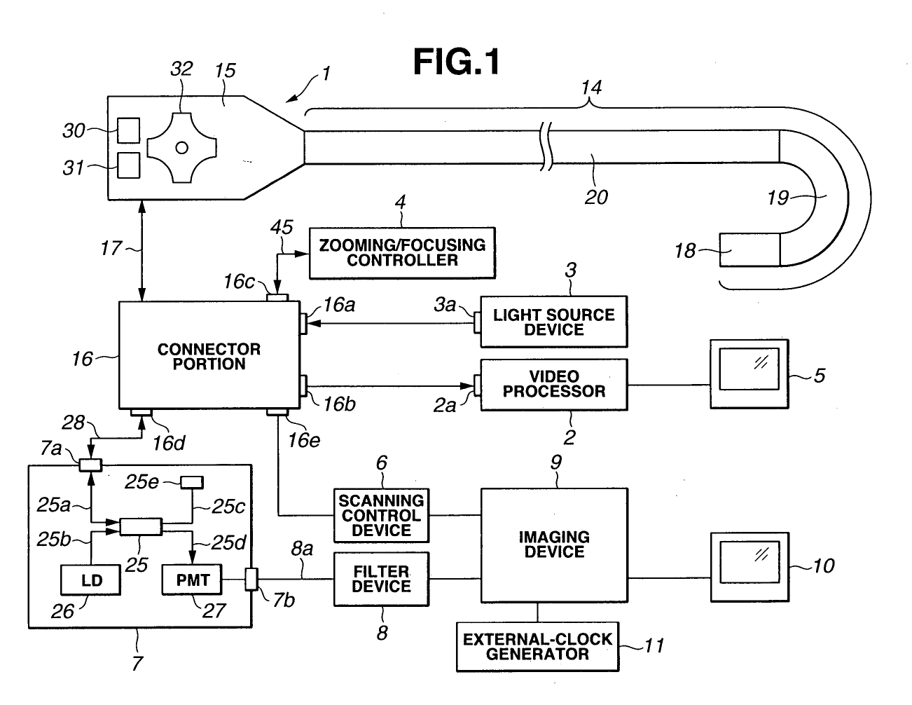

FIGS. 1 to 4 show a first embodiment of the present invention.

An endoscope apparatus according to the first embodiment comprises: an endoscope 1; a video processor 2; a light source device 3; a zooming/focusing controller 4 which controls the zooming and the focus of a microscopic observing optical system 37 (refer to FIG. 3) arranged to an edge portion 18 of the endoscope 1 (hereinafter, referred to as an endoscope edge-portion 18) which will be described later; and an endoscope observation monitor 5 which is connected to the video processor 2 and displays an observing image.

Further, the endoscope apparatus comprises: a scanning control device 6 which drives a scanner 37b arranged to the microscopic observing optical system 37; an optical unit 7 which supplies light to the microscopic observing optical system 37, detects an optical image from the microscopic observing optical system 37, and converts the image into an electrical signal; a filter device 8 through which only a specific frequency component of the electrical signal from the optical unit 7 is passed; an imaging device 9 which images the electrical signal from the filter device 8; a microscopic observation image monitor 10 which displays a video signal from the imaging device 9; and an external-clock generator 11 which generates a clock as a reference of a driving waveform for driving the scanner 37b.

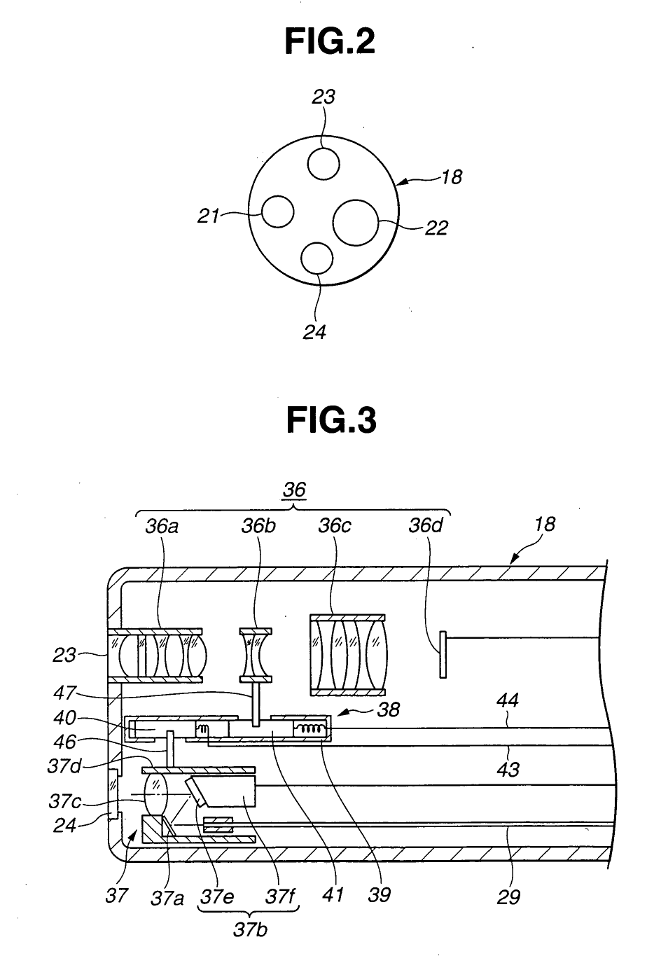

The endoscope 1 comprises: an inserting portion 14; an operating portion 15 which is continuously arranged to the rear end of the inserting portion 14; a connector portion 16; and a connecting cord 17 which connects the operating portion 15 and the connector portion 16. The inserting portion 14 has, from the edge side, the endoscope edge-portion 18, a bending portion 19 which is freely bent, and a soft flexible tube portion 20. Further, referring to FIG. 2, arranged to the edge surface of the endoscope edge-portion 18 in the endoscope 1 are an illuminating window 21 for endoscope observation, a treatment-tool channel opening portion 22, an endoscope observing window 23, and a microscopic observing window 24.

The optical unit 7 has a four-terminal coupler 25 having four end-portions of first to fourth end-portions 25a to 25d, a microscopic observation laser diode 26 (hereinafter, referred to as a microscopic observation LD) 26 as a light source, and a photo multiplier tube (hereinafter, referred to as a PMT) unit 27.

The first end-portion 25a in the four-terminal coupler 25 is optically connected to an optical fiber 28 via a connector 7a. The second end-portion 25b is optically connected to the microscopic observation LD 26. The third end-portion 25c is terminated by an optical fiber terminal end 25e. The fourth end-portion 25d is optically connected to the PMT unit 27. The PMT unit 27 is electrically connected to the filter device 8 via a connector 7b and a signal line 8a.

At the four-terminal coupler 25, the light inputted from the second end-portion 25b and the fourth end-portion 25d is branched to the first end-portion 25a and the third end-portion 25c. On the contrary, the light inputted from the first end-portion 25a and the third end-portion 25c is branched to the second end-portion 25b and the fourth end-portion 25d. Laser beams generated by the microscopic observation LD 26 are outputted to the connector portion 16 side in the endoscope 1 via the second end-portion 25b, the four-terminal coupler 25, and the first end-portion 25a.

The connector portion 16 includes a first connector 16a which guides illumination light for endoscope observation, a second connector 16b which sends, to the video processor 2, a signal from an image pick-up unit 36, as will be described later, a third connector 16c which receives and transmits a signal for controlling a linear actuator mover 41 for adjusting an angle of view and a linear actuator mover 40 for adjusting a focal point that are arranged to the endoscope edge-portion 18, as will be described later, a fourth connector 16d which inputs light transmitted via an optical fiber 29 (refer to FIG. 3) inserted in the inserting portion 14 of the endoscope 1 to the microscopic observing optical system 37, and a fifth connector 16e which receives and transmits a signal for controlling the scanner 37b in the microscopic observing optical system 37.

A connecting relationship among the connectors 16a to 16d will be described here. The first connector 16a is detachably connected to a socket 3a in the light source device 3. The second connector 16b is detachably connected to a socket 2a in the video processor 2 via a curling cord. Further, the third connector 16c is detachably connected to the zooming/focusing controller 4 via a cord. The fourth connector 16d is optically connected to the optical unit 7. The fifth connector 16e in the connector portion 16 is detachably connected to the scanning control device 6.

Laser beams are generated by the microscopic observation LD 26 and are inputted to the fourth connector 16d arranged to the connector portion 16 in the endoscope 1 via the connector 7a and the optical fiber 28 from the first end-portion 25a in the four-terminal coupler 25. The laser beams pass through the optical fiber 29 which is connected via the connecting cord 17 and which is arranged to the endoscope edge-portion 18 from the operating portion 15 in the endoscope 1, and are transmitted to the microscopic observing optical system 37 arranged to the endoscope edge-portion 18.

The operating portion 15 in the endoscope 1 has a zooming switch 30 which changes an angle of view for endoscope observation and a focusing switch 31 which moves the focusing position of the microscopic observing optical system 37. Reference numeral 32 denotes a bending knob which bends the bending portion 19 in an arbitrary direction. The zooming switch 30 and the focusing switch 31 are connected from the connecting cord 17 and the connector portion 16 to the zooming/focusing controller 4 via the third connector 16c arranged to the connector portion 16.

Next, a description is given of the internal structure of the endoscope edge-portion 18. Referring to FIG. 3, the image pick-up unit 36, the microscopic observing optical system 37, and a linear actuator 38 are arranged at predetermined position in the shaft-core direction of the endoscope edge-portion 18 therein. Further, a light guide for illumination and a treatment tool channel which are not shown are arranged in the endoscope edge-portion 18.

In the image pick-up unit 36, as an optical system for endoscope observation, a first lens-group 36a, a second lens-group 36b, and a third lens-group 36c are sequentially arranged from the edge side of the inserting portion 14 in the endoscope 1. In the back thereof, the solid image pick-up device 36d is arranged. The first lens-group 36a is fixed to the endoscope observing window 23.arranged to the edge surface of the endoscope edge-portion 18. The second lens-group 36b functions as a zooming optical system which can freely move between the first lens-group 36a and the third lens-group 36c. The second lens-group 36b is close to the first lens-group 36a side and then the angle of view for observation is gradually increased. On the contrary, the second lens-group 36b is apart from the first lens-group 36a and then the angle of view for observation is gradually decreased.

The microscopic observing optical system 37 comprises a first mirror 37a which reflects the light outputted from the end portion of the optical fiber 29, the scanner 37b, and the objective optical system 37c. The objective optical system 37c is held to a mirror frame 37d. The scanner 37b has a second mirror 37e and driving means 37f such as a motor which drives the second mirror 37e at a constant speed. The scanner 37b scans the specimen in the two-dimensional direction (X-axis and Y-axis directions) perpendicular to the optical axis (Z-axis direction) by the change in reflecting direction in accordance with the rotation of the second mirror 37e.

The light reflected from the specimen passes through a contrary route and reaches the four-terminal coupler 25 arranged to the optical unit 7. Further, the light passes through the third end-portion 25c and is transmitted to the PMT unit 27. Then, an optical signal is converted into an electrical signal and the electrical signal as a result of the photo-electrical conversion is transmitted to the filter device 8 via the connector 7b and the signal line 8a.

The linear actuator 38 drives the second lens-group 36b forming the image pick-up unit 36 and the mirror frame 37d forming the microscopic observing optical system 37 in the optical axis direction. A cylindrical guide member 39 forming the linear actuator 38 is arranged in the axial direction of the endoscope edge-portion 18 in parallel therewith between the image pick-up unit 36 and the microscopic observing optical system 37.

Arranged to the guide member 39 in the linear actuator 38 in front and back on the same axis are the linear actuator mover 40 for adjusting the focal point as a focal-point adjusting mechanism and the linear actuator mover 41 for adjusting the angle of view as a moving mechanism for adjusting the angle of view. Further, the linear actuator mover 40 for adjusting the focal point and the linear actuator mover 41 for adjusting the angle of view are movably supported to the inner periphery of the guide member 39.

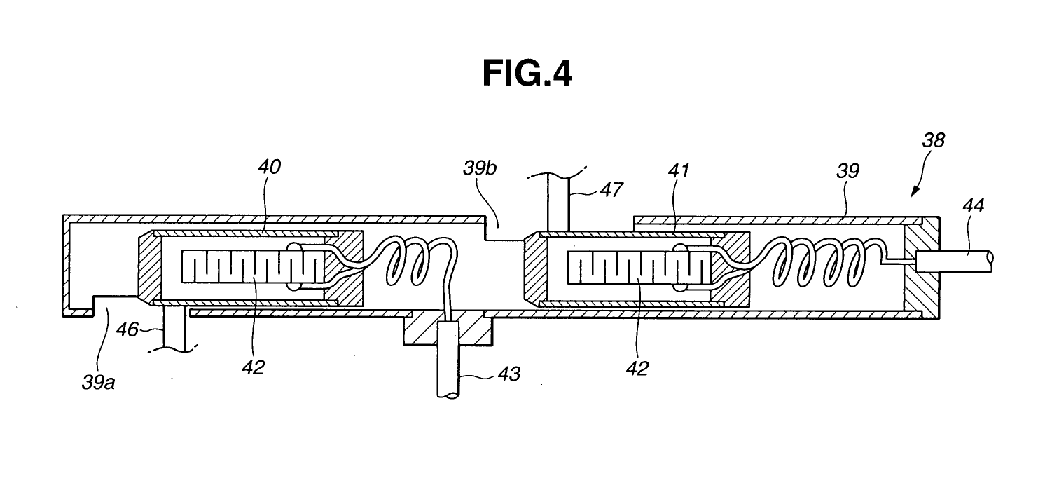

According to the first embodiment, an impact-type piezoelectric actuator (fast-modifying actuator) is used as the linear actuator mover 40 for adjusting the focal point and the linear actuator mover 41 for adjusting the angle of view. Referring to FIG. 4, the linear actuator mover 40 for adjusting the focal point and the linear actuator mover 41 for adjusting the angle of view include piezoelectric elements 42, respectively. The piezoelectric element 42 is formed by laminating, along the axial direction, piezoelectric members such as barium titanate, lead zirconate titanate, and ceramics including porcelain. Leads 43 and 44 for supplying driving force to electrodes arranged to the piezoelectric elements 42 are connected to the linear actuator mover 40 for adjusting the focal point and the linear actuator mover 41 for adjusting the angle of view, respectively. The leads 43 and 44 are extended to the third connector 16c arranged to the connector portion 16 in the endoscope 1, and are electrically connected to the zooming/focusing controller 4 via a zooming/focusing cable 45.

The linear actuator mover 40 for adjusting the focal point and the linear actuator mover 41 for adjusting the angle of view are held in a state in which they are pressed and come into contact with the inner periphery of the guide member 39 by using a friction member (not shown). A driving voltage with a predetermined waveform is applied to the piezoelectric elements 42 from the zooming/focusing controller 4 and the piezoelectric elements 42 are mechanically expanded or contracted. The expansion and contraction shocks the linear actuator mover 40 for adjusting the focal point and the linear actuator mover 41 for adjusting the angle of view. The shock over the friction force caused by the friction member operates forward and backward the linear actuator mover 40 for adjusting the focal point and the linear actuator mover 41 for adjusting the angle of view.

In the halfway of the guide member 39, notch portions 39a and 39b are formed. The linear actuator mover 40 for adjusting the focal point and the linear actuator mover 41 for adjusting the angle of view are exposed from the notch portions 39a and 39b. The exposed linear actuator mover 40 for adjusting the focal point and the exposed linear actuator mover 41 for adjusting the angle of view are connected to the mirror frame 37d in the microscopic observing optical system 37 and the second lens-group 36b in the image pick-up unit 36 via a connecting member 46 for adjusting the focal point and a connecting member 47 for moving the angle of view.

Therefore, the mirror frame 37d in the microscopic observing optical system 37 and the second lens-group 36b in the image pick-up unit 36 trace the advance and return operations of the linear actuator mover 40 for adjusting the focal point and the linear actuator mover 41 for adjusting the angle of view in the optical axis direction, and move in the same direction as those of the linear actuator mover 40 for adjusting the focal point and the linear actuator mover 41 for adjusting the angle of view. The advance and return operation of the mirror frame 37d in the microscopic observing optical system 37 adjusts the focusing operation, and the advance and return operation of the second lens-group 36b changes the angle of view of the observing image for the endoscope.

Next, a description is given of the operations with the above-mentioned structure according to the first embodiment.

In the endoscope observation, illumination light outputted from the light source device 3 passes through a light guide (not shown) inserted into the endoscope 1 and is outputted from the illuminating window 21 for endoscope observation arranged to the edge surface of the endoscope edge-portion 18, thereby illuminating a subject.

Reflecting light from the subject is inputted via the first lens-group 36a, the second lens-group 36b, and the third lens-group 36c. A subject image is formed onto an image pick-up surface of the solid image pick-up device 36d. The subject image formed onto the image pick-up surface is converted into an electrical signal, and is transmitted to the video processor 2. The video processor 2 converts the electrical signal into a video signal by an incorporated processing circuit (not shown), and is outputted to the endoscope observation monitor 5. Then, the subject image is displayed on the endoscope observation monitor 5.

In the case of the endoscope observation using the endoscope 1, the zooming switch 30 arranged to the operating portion 15 in the endoscope 1 is switched to an enlargement side or a wide-angle-of-view side. Then, a corresponding signal is inputted to the zooming/focusing controller 4. The zooming/focusing controller 4 outputs a driving signal to the linear actuator mover 41 for adjusting the angle of view via the lead 44 based on the signal from the zooming switch 30.

Then, the linear actuator mover 41 for adjusting the angle of view advances and returns in the guide member 39 by using the shock caused by the mechanical expansion and contraction of the incorporated piezoelectric element 42. Since the second lens-group 36b forming the image pick-up unit 36 is continuously arranged to the linear actuator mover 41 for adjusting the angle of view via the connecting member 47 for moving the angle of view, the second lens-group 36b moves integrally with the linear actuator mover 41 for adjusting the angle of view in the same direction as that of the linear actuator mover 41 for adjusting the angle of view. Then, the movement of the second lens-group 36b in the close direction to the first lens-group 36a gradually increases the angle of view for observation, and the subject image displayed on the endoscope observation monitor 5 is enlarged. On the other hand, the movement of the second lens-group 36b apart from the first lens-group 36a gradually decreases the angle of view for observation, the subject image displayed on the endoscope observation monitor 5 has a wide angle of view.

When the concerned portion such as the lesion portion during the endoscope observation, the microscopic observation starts.

In the microscopic observation, the scanning control device 6 is driven and then the scanner 37b arranged to the microscopic observing optical system 37 is driven by the scanning control device 6. Next, the microscopic observation LD 26 arranged to the optical unit 7 starts. The light from the microscopic observation LD 26 is scanned in the two-dimensional direction (X-axis direction and Y-axis direction) perpendicular to the optical axis (Z-axis direction) near the surface of the concerned portion. A scanning range in this case is set with a square area of 300 μm according to the first embodiment. However, the present invention is not limited to this.

The reflecting light from the concerned portion is inputted to the PMT unit 27 arranged to the optical unit 7 via the optical fiber 29, and is converted into an electrical signal. The electrical signal as a result of the photoelectric conversion of the PMT unit 27 is transmitted to the filter device 8 via the signal line 8a connected to the output side of the PMT unit 27 via the connector 7b. Further, the electrical signal through the filter device 8 is inputted to the imaging device 9 and is imaged. Then, the signal is inputted to the microscopic observation image monitor 10 and the image of the concerned portion as the microscopic observation is displayed on the microscopic observation image monitor 10.

When the image as the result of microscopic observation displayed on the microscopic observation image monitor 10 is not focused, or when the observation target moves and the image is not focused, an operator operates the focusing switch 31 arranged to the operating portion 15 in the endoscope 1. Then, a corresponding focusing signal is transmitted to the zooming/focusing controller 4.

The zooming/focusing controller 4 outputs the driving signal to the linear actuator mover 40 for adjusting the focal point via the lead 43 based on the focusing signal from the focusing switch 31. The linear actuator mover 40 for adjusting the focal point moves in a predetermined direction. The microscopic observing optical system 37, which is connected to the linear actuator mover 40 for adjusting the focal point via the connecting member 46 for adjusting the focal point, moves in the same direction as that of the linear actuator mover 40 for adjusting the focal point. Thus, the image is focused to the concerned portion.

According to the first embodiment, the linear actuator mover 41 for adjusting the angle of view and the linear actuator mover 40 for adjusting the focal point are arranged on the same axis in the front and back direction to the one guide member 39 and therefore the space in the diameter direction of the endoscope edge-portion 18 in the endoscope 1 is reduced and the diameter of the endoscope edge-portion 18 is made thin.

(Second Embodiment)

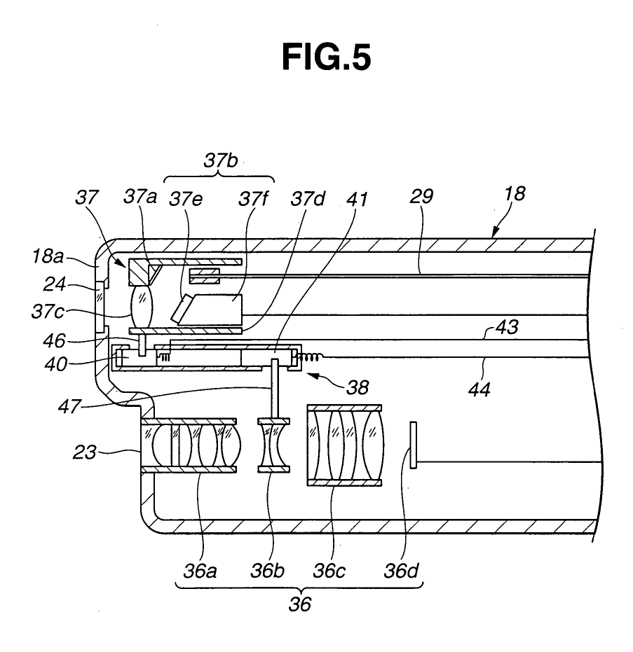

FIGS. 5 to 7 show the second embodiment of the present invention.

According to the first embodiment, the edge surface of the endoscope edge-portion 18 is flat. However, according to the second embodiment, a projected portion 18a having a projecting amount L is formed onto the edge surface of the endoscope edge-portion 18, and the microscopic observing window 24 is arranged to the projected portion 18a.

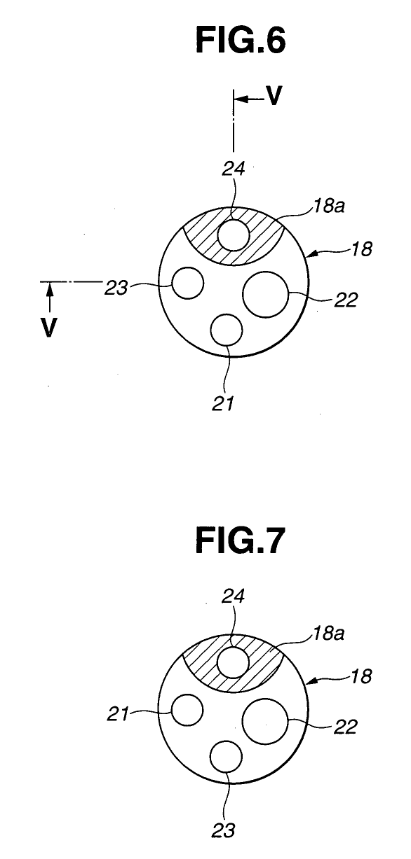

Referring to FIG. 6, the edge surface of the endoscope edge-portion 18 has not only the microscopic observing window 24 arranged to the projected portion 18a (hatched in the drawing), but also the illuminating window 21 for endoscope observation at the position apart from the microscopic observing window 24 by approximately 180° as a center angle. Further, the endoscope observing window 23 and the treatment-tool channel opening portion 22 are arranged in the left and right directions in view of the edge side.

Referring to FIG. 5, since the microscopic observing window 24 is projected from the endoscope observing window 23 by the projecting amount L, the microscopic observing optical system 37 arranged backward of the microscopic observing window 24 is projected, by the projecting amount L, in front of the image pick-up unit 36 arranged backward of the endoscope observing window 23.

The projecting amount L is set so that the focusing position on the subject side in a maximum zooming state of the image pick-up unit 36 matches the edge surface of the microscopic observing window 24. In other words, the projecting amount L is set so that it matches a WD (working distance) in the maximum zooming state of the image pick-up unit 36.

With the above-mentioned structure, the projected portion 18a is formed to the edge surface of the endoscope edge-portion 18 and the projecting amount L of the projected portion 18a is set so that it matches the WD (working distance) in the maximum zoom of the image pick-up unit 36. Thus, when the edge surface of the projected portion 18a is pressed to the subject during the microscopic observation, the image is focused in the maximum zoom state of the image as the result of endoscope observation. Although the observing position is slightly varied, it is easily identified from the subject image displayed on the endoscope observation monitor 5, which portion is microscopically observed and thus it is convenient.

The illuminating window 21 for endoscope observation is arranged at the position apart from the microscopic observing window 24 by approximately 180° as a center angle, namely, at the farthest position. Consequently, the illumination light for endoscope observation is hardly inputted to the microscopic observing optical system 37 and it is prevented that the illumination light for endoscope observation becomes noises against the microscopic observing optical system 37.

In this case, the endoscope observing window 23 is arranged at the position apart from the microscopic observing window 24 by approximately 180° as a center angle, namely, at the farthest position. The illuminating window 21 for endoscope observation and the treatment-tool channel opening portion 22 are arranged in the left and right directions of the edge surface, the laser beams outputted from the microscopic observing window 24 are hardly inputted to the image pick-up unit 36 from the endoscope observing window 23 and it is prevented that the laser beams become noises against the image pick-up unit 36.

(Third Embodiment)

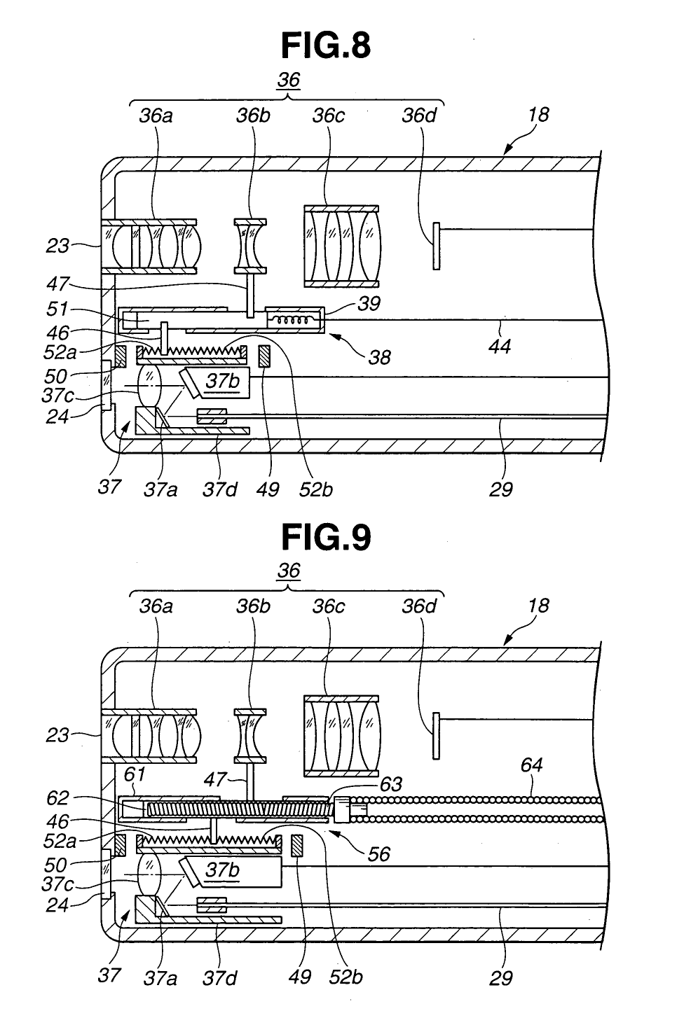

FIG. 8 shows the third embodiment of the present invention.

According to the first embodiment, the linear actuator mover 40 for adjusting the focal point and the linear actuator mover 41 for adjusting the angle of view are individually supported to the guide member 39. However, according to the third embodiment, one linear actuator mover 51 for adjusting the focal point and the angle of view performs the same operation as those of the linear actuator mover 40 for adjusting the focal point and the linear actuator mover 41 for adjusting the angle of view.

That is, the linear actuator mover 51 for adjusting the focal point and the angle of view has the same structure as those of the linear actuator mover 40 for adjusting the focal point or the linear actuator mover 41 for adjusting the angle of view. A lead 48 connected to the linear actuator mover 51 for adjusting the focal point and the angle of view is extended to the third connector portion 16c arranged to the connector portion 16 in the endoscope 1 shown in FIG. 1 and it is electrically connected to the zooming/focusing controller 4 via the zooming/focusing cable 45.

The operator operates the zooming switch 30 or the focusing switch 31 arranged to the operating portion 15 in the endoscope 1. Then, a corresponding zooming signal or focusing signal is transmitted to the zooming/focusing controller 4. A driving signal is outputted to the linear actuator mover 51 for adjusting the focal point and the angle of view from the zooming/focusing controller 4 via the lead 48.

In general, the amount of movement for moving the second lens-group 36b in the image pick-up unit 36 is necessary for changing the angle of view for observation using the endoscope and is larger than the amount of movement for moving the microscopic observing optical system 37 so as to focus the microscopic observing optical system 37 during the microscopic observation.

Therefore, when the one linear actuator mover 51 for adjusting the focal point and the angle of view drives both the second lens-group 36b and the microscopic observing optical system 37, a moving range of the second lens-group 36b needs to be larger than a moving range of the microscopic observing optical system 37.

According to the third embodiment, a connecting member 46 for adjusting the focal point extended from the linear actuator mover 51 for adjusting the focal point and the angle of view is not directly fixed to the mirror frame 37d in the microscopic observing optical system 37, and is sandwiched by a pair of opposed compressing springs 52a and 52b. With the above-mentioned structure, when the linear actuator mover 51 for adjusting the focal point and the angle of view moves over the moving range of the microscopic observing optical system 37, the connecting member 46 for adjusting the focal point and the mirror frame 37d in the microscopic observing optical system 37 allow to be moved relatively by the expanding and contracting operation of the pair of the opposed compressing springs 52a and 52b as elastic members.

That is, the endoscope edge-portion 18 has a first stopper 49 and a second stopper 50 which mechanically restricts the moving range of the mirror frame 37d arranged to the microscopic observing optical system 37. The mirror frame 37d comes into contact with one of the first stopper 49 and the second stopper 50, thereby limiting the moving range. The first stopper 49 and the second stopper 50 restrict the moving range of the microscopic observing optical system 37, and the moving range of the microscopic observing optical system 37 which is restricted by the first stopper 49 and the second stopper 50 is set to be within the moving range of the second lens-group 36b.

Next, the operations with the above-mentioned structure will be described according to the third embodiment. The same operations as those according to the first embodiment are not described here.

When the angle of view for observation changes during the endoscope observation, the zooming switch 30 arranged to the operating portion 15 (refer to FIG. 1) in the endoscope 1 is operated, thereby moving the linear actuator mover 51 for adjusting the focal point and the angle of view inserted in the guide member 39 in the linear actuator 38. Then, the angle of view for observation in the endoscope 1 changes by moving the second lens-group 36b connected to the linear actuator mover 51 for adjusting the focal point and the angle of view via the connecting member 47 for moving the angle of view.

The microscopic observing optical system 37 is connected to the linear actuator mover 51 for adjusting the focal point and the angle of view via the connecting member 46 for adjusting the focal point. The connecting member 46 for adjusting the focal point is sandwiched between the pair of the compressing springs 52a and 52b and is supported to the mirror frame 37d in the microscopic observing optical system 37. Therefore, the front end or back end of the mirror frame 37d does not come into contact with the first stopper 49 or the second stopper 50. In the movement allowed area, the connecting member 46 for adjusting the focal point maintains to be neutral by the compressing springs 52a and 52b and therefore the microscopic observing optical system 37 moves in the same direction as that of the second lens-group 36b synchronously therewith.

After that, the front end or back end of the mirror frame 37d comes into contact with the first stopper 49 or the second stopper 50 and then the movement of the mirror frame 37d stops. However, the movement of the connecting member 46 for adjusting the focal point is allowed by the expansion and contraction operation of the pair of the compressing springs 52a and 52b, and the connecting member 46 for adjusting the focal point moves integrally with the linear actuator mover 51 for adjusting the focal point and the angle of view.

Even after the mirror frame 37d comes into contact with the first stopper 49 or the second stopper 50, the amount of displacement of the compressing springs 52a and 52b, an interval between the first stopper 49 and the front end of the mirror frame 37d, and an interval between the second stopper 50 and the rear end of the mirror frame 37d are set to have values so that the second lens-group 36b is moved up to the maximum moving range without trouble.

Next, when the focal point of the microscopic observing optical system 37 is adjusted in the observation switched from the endoscope observation to the microscopic observation, the focusing switch 31 arranged to the operating portion 15 in the endoscope 1 is operated.

Then, the linear actuator mover 51 for adjusting the focal point and the angle of view moves along the guide member 39, and the connecting member 46 for adjusting the focal point extended from the linear actuator mover 51 for adjusting the focal point and the angle of view moves the mirror frame 37d via the pair of the compressing springs 52a and 52b which holds the connecting member 46 for adjusting the focal point in a neutral state. In this state, the moving range of the mirror frame 37d is set to an area where it does not come into contact with the first stopper 49 and the second stopper 50. Therefore, the mirror frame 37d is moved in a state in which it traces the linear actuator mover 51 for adjusting the focal point and the angle of view. In this case, the second lens-group 36b traces to the linear actuator mover 51 for adjusting the focal point and the angle of view.

According to the third embodiment, the one linear actuator mover 51 for adjusting the focal point and the angle of view moves both the second lens-group 36b in the image pick-up unit 36 and the mirror frame 37d in the microscopic observing optical system 37. Thus, the number of parts is reduced and the space is saved.

(Fourth Embodiment)

FIG. 9 shows the fourth embodiment of the present invention.

According to the third embodiment, the linear actuator mover 51 for adjusting the focal point and the angle of view using the piezoelectric elements moves both the second lens-group 36b in the image pick-up unit 36 and the mirror frame 37d in the microscopic observing optical system 37. However, according to the fourth embodiment, a feed-screw mechanism 56 for rotation moves the second lens-group 36b in the image pick-up unit 36 and the mirror frame 37d in the microscopic observing optical system 37. The fourth embodiment is a modification of the third embodiment and therefore the same components are designated by the same reference numerals as those according to the third embodiment and the description thereof is omitted.

In the endoscope edge-portion 18, a guide member 61 is arranged between the image pick-up unit 36 and the microscopic observing optical system 37 in parallel with the endoscope edge-portion 18 along the axial direction thereof. A direct driven member 62 is inserted in the guide member 61 in a state in which only the advance and return operation of the direct driven member 62 is permitted by spline engagement or the like. The connecting member 46 for adjusting the focal point and the connecting member 47 for moving the angle of view are fixed to the direct driven member 62.

Further, a feed screw 63 is screwed in the direct driven member 62, and the edge of a flexible shaft 64 is fixed to the rear end of the feed screw 63. The flexible shaft 64 is inserted in the inserting portion 14 (refer to FIG. 1) in the endoscope 1, and the rear end thereof is arranged to a rotating motor (not shown) arranged near the operating portion 15 (refer to FIG. 1) in the endoscope 1.

The zooming switch 30 or the focusing switch 31 (refer to FIG. 1) arranged to the operating portion 15 is operated, thereby driving the rotating motor. Then, the rotation of a spindle of the rotating motor is transmitted to the feed screw 63 via the flexible shaft 64.

The feed screw 63 is screwed into the direct driven member 62, and the direct driven member 62 is inserted in the guide member 61 in a state in which only the advance and return operation thereof is permitted by the spline engagement. Thus, the rotation of the feed screw 63 advances and returns the direct driven member 62.

As a result, the direct driven member 62 advances and returns, similarly to the linear actuator mover 51 for adjusting the focal point and the angle of view according to the third embodiment. Further, the image pick-up unit 36 is zoomed and the focal point of the microscopic observing optical system 37 is adjusted.

According to the fourth embodiment, the rotating motor as a driving source is arranged on the operating portion 15 side in the endoscope 1 and therefore, the rotating motor having a large capacity can be used. Further, large driving force is easily obtained. Since the feed-screw mechanism is arranged to the endoscope edge-portion 18 and the feed-screw mechanism moves the second lens-group 36b in the image pick-up unit 36 and the mirror frame 37d in the microscopic observing optical system 37, the actuator is not necessary for the endoscope edge-portion 18. Further, the countermeasure against the heating is not necessary for the actuator and therefore the structure becomes simple.

According to the fourth embodiment, the second lens-group 36b in the image pick-up unit 36 and the mirror frame 37d in the microscopic observing optical system 37 are moved by using the feed-screw mechanism. However, the feed-screw mechanism is substituted with a mechanism for converting the rotating movement into the linear movement, such as a cam link mechanism. As long as the rotating motor generates rotating force, the actuator other than the rotating motor can be used.

As mentioned above, according to the first to fourth embodiments, the one endoscope includes the optical system for endoscope observation and the optical system for microscopic observation, and the moving mechanism for adjusting the angle of view which changes the angle of view by moving a part of the optical system for endoscope observation and the focal-point adjusting mechanism which moves the focal point on the subject side in the optical system for microscopic observation. Further, the moving mechanism for adjusting the angle of view and the focal-point adjusting mechanism are arranged to the inserting portion forward and backward on the same axis. Therefore, the space in the edge of the inserting portion is saved and the enlargement magnification of the endoscope image can be modified and the focal point of the confocal image can be adjusted by using one endoscope.

Further, since the endoscope observation and the microscopic observation are switched and are operated in the single endoscope, the focal point in the microscopic observation can be adjusted precisely, microscopic images can be easily obtained even when the subject moves and the convenience is excessively improved.

(Fifth Embodiment)

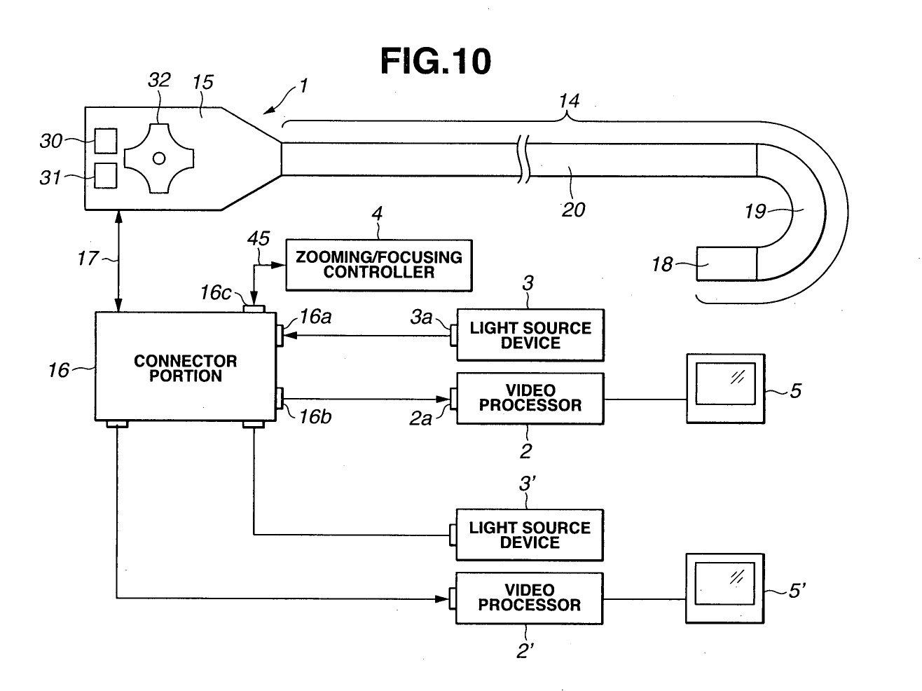

FIGS. 10 to 14 show the fifth embodiment of the present invention. The fifth embodiment is a modification of the first embodiment and the same components as those according to the first embodiment are designated by the reference numerals. The description thereof is omitted here.

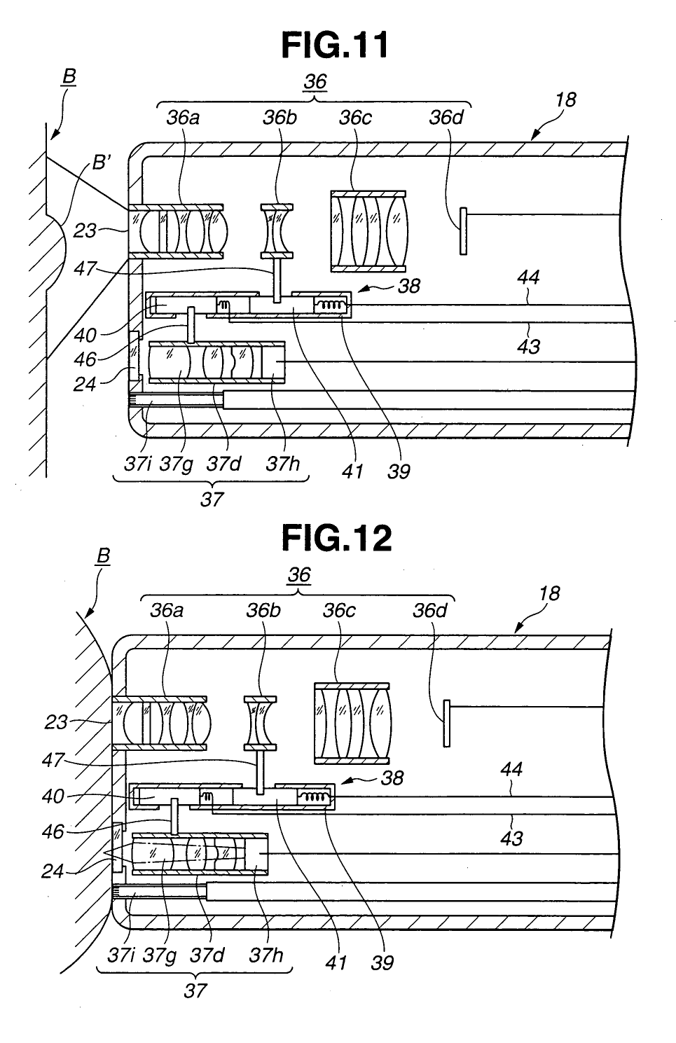

Referring to FIGS. 11 and 12, according to the fifth embodiment, the mirror frame 37d in the microscopic observing optical system 37 arranged to the endoscope edge-portion 18 holds, from the microscopic observing window 24 side, a high-magnification lens group 37g having a short focusing distance and a large aperture, and a solid image pick-up device 37h for microscopic observation. Further, a light guide 37i for microscopic observation is arranged, as illuminating means, near the mirror frame 37d in parallel therewith.

Referring to FIG. 10, an endoscope observing apparatus comprises a video processor 2′ which drives the solid image pick-up device 37h arranged to the microscopic observing optical system 37, a light source device 3′ which is connected to the light guide 37i and supplies illumination light to the light guide 37i, and an endoscope observation monitor 5′ which is connected to the video processor 2′ and displays a microscopic observing image that is picked up by the solid image pick-up device 37h.

The video processor 2′, the light source device 3′, and the endoscope observation monitor 5′ which are used for the microscopic observation may be shared with the video processor 2, the light source device 3, and the endoscope observation monitor 5 which are used for the endoscope observation.

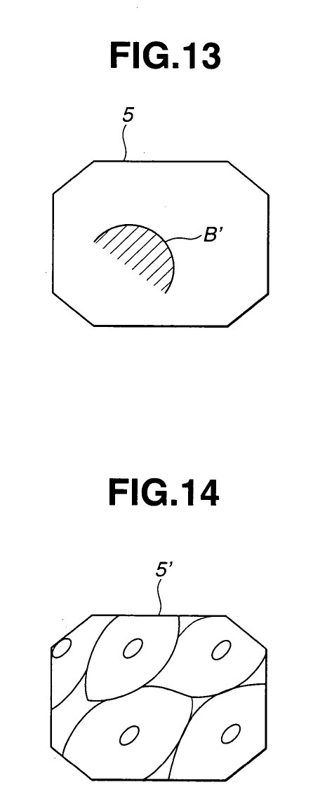

With the structure, when a living body B is observed by the endoscope, similarly to the first embodiment, the reflecting light from the subject is received by the solid image pick-up device 36d via the image pick-up unit 36 and the subject image is displayed on the endoscope observation monitor 5 (refer to FIG. 13).

In this case, the endoscope observation monitor 5 enlarges and displays, or displays with a wide angle of view, a concerned portion B′ such as the lesion portion by moving the linear actuator mover 41 for adjusting the angle of view arranged to the linear actuator 38.

When the concerned portion B′ is microscopically observed, the endoscope edge-portion 18 is touched to the living body B. Light is outputted from the light guide 37i for microscopic observation and is turned in the living body B. The concerned portion B′ is microscopically observed by illuminating the light. The observing range of the microscopic observing optical system 37 is 0 to 100 μm from the microscopic observing window 24.

According to the fifth embodiment, the living body B to be observed is stained by a dye that is generally used for the endoscope observation, such as methylene blue. Then, the living body B is cleaned and is touched to the endoscope edge-portion 18, thereby observing the living body B.

In this case, the movement of the linear actuator mover 40 for adjusting the focal point arranged to the linear actuator 38 drives, in the optical axis direction, the mirror frame 37d which integrally holds the high-magnification lens group 37g as the high-magnification enlargement optical system and the solid image pick-up device 37h as the image pick-up means, thereby adjusting the focal point for the microscopic observation.

As a result, referring to FIG. 14, the endoscope observing monitor 5′ displays the subject image which is enlarged by 500 to 1000 times and, therefore, the structures of cell and gland are observed by using the subject image.

Referring to FIG. 8, the linear actuator 38 may be operated by the linear actuator mover 51 for adjusting the focal point and the angle of view. Alternatively, referring to FIG. 9, the linear actuator 38 may be operated by the feed-screw mechanism 56 for rotation.

(Sixth Embodiment)

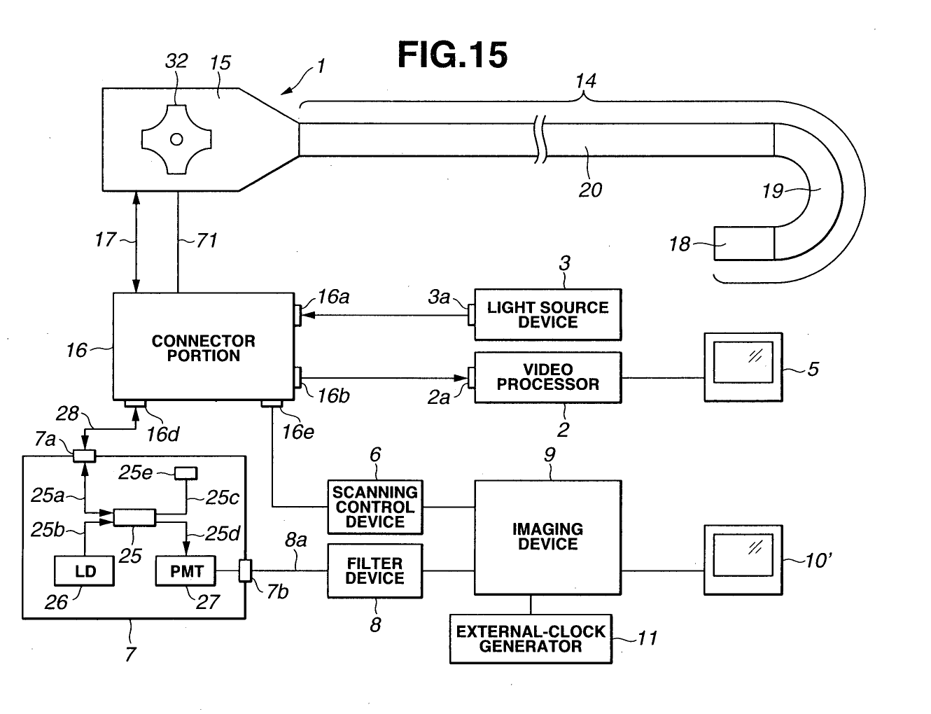

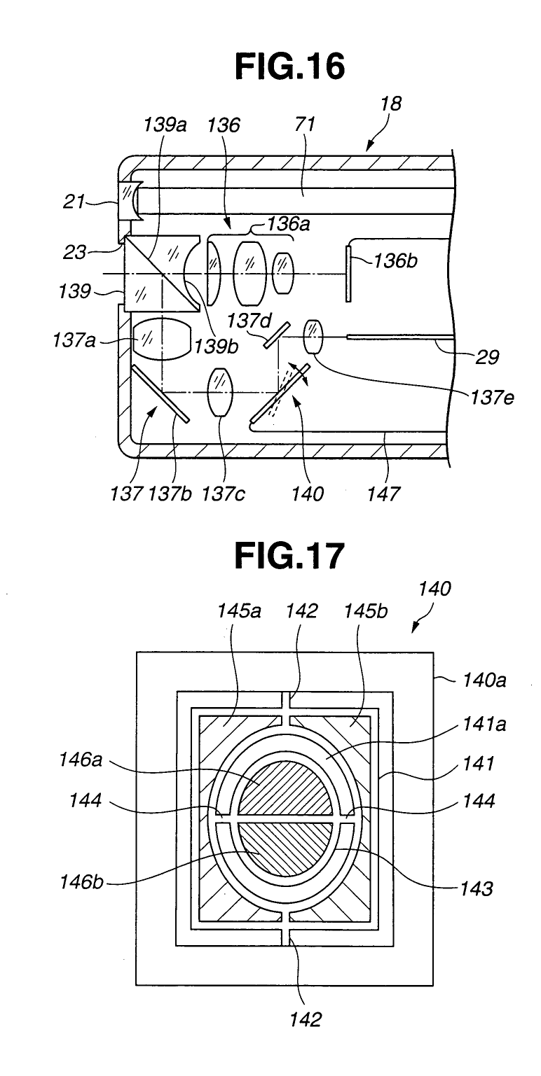

FIGS. 15 to 17 show the sixth embodiment of the present invention. FIG. 15 is a diagram showing the structure of an endoscope apparatus. FIG. 16 is a cross-sectional view showing an edge portion of an endoscope. FIG. 17 is a front view showing a scanning mirror.

Referring to FIG. 15, an endoscope apparatus according to the sixth embodiment comprises: the endoscope 1; and the normal observation system and the confocal observation system which are connected to the endoscope 1.

The normal observation system comprises the endoscope 1, the video processor 2, the light source device 3, and the endoscope observation monitor 5 which is connected to the video processor 2 and displays the subject image.

The confocal observation system comprises: the scanning control device 6 which is arranged to a confocal optical system 137, as will be described later, and drives a scanning mirror 140; the optical unit 7 which supplies light to the confocal optical system 137 with a high resolution, detects an optical image from the confocal optical system 137, and converts the image into an electrical signal; the filter device 8 through which only a specific frequency component of a signal from the optical unit 7 passes; the imaging device 9 which images the electrical signal from the filter device 8; a confocal image monitor 10′ which displays a video signal from the imaging device 9; and the external-clock generator 11 which generates a clock pulse as a reference of a driving waveform for driving the scanning mirror 140.

The endoscope 1 comprises the inserting portion 14, the operating portion 15 which is continuously arranged to the rear end of the inserting portion 14, the connector portion 16, and the connecting cord 17 which connects the operating portion 15 and the connector portion 16.

The inserting portion of the endoscope 1 (hereinafter, referred to as an “endoscope inserting portion”) 14 comprises, from the edge side, the endoscope edge-portion 18, the bending portion 19 which can freely be bent, and the soft flexible tube portion 20. Further, referring to FIG. 16, the edge surface of the edge portion 18 of the endoscope 1 (hereinafter, referred to as the “endoscope edge-portion”) has, as opening portions, the illuminating window 21 for endoscope observation which projects visible light for normal observation and the endoscope observing window 23.

The optical unit 7 comprises the four-terminal coupler 25 having the first to fourth end-portions 25a to 25d, and the confocal observation laser diode 26 (hereinafter, referred to as confocal observation LD 26) as a light source which emits infrared light, and the photo multiplier tube (hereinafter, referred to as the PMT unit) 27. The first end-portion 25a in the four-terminal coupler 25 is optically connected to an optical fiber 28 via the connector 7a. The second end-portion 25b is optically connected to the confocal observation LD 26. The third end-portion 25c is terminated by the optical fiber terminal end 25e. The fourth end-portion 25d is optically connected to the PMT unit 27. The PMT unit 27 is electrically connected to the filter device 8 via the connector 7b and the signal line 8a.

At the four-terminal coupler 25, the light inputted from the second end-portion 25b and the fourth end-portion 25d is branched to the first end-portion 25a and the third end-portion 25c. On the contrary, the light inputted from the first end-portion 25a and the third end-portion 25c is branched to the second end-portion 25b and the fourth-end portion 25d. Laser beams generated by the confocal observation LD 26 are outputted to the connector portion 16 side in the endoscope 1 via the second end-portion 25b, the four-terminal coupler 25, and the first end-portion 25a.

The connector portion 16 includes: the first connector 16a which guides visible light for endoscope observation; the second connector 16b which sends, to the video processor 2, a signal from the image pick-up unit 36, as will be described later; the fourth connector 16d which inputs, to the confocal optical system 137, light transmitted via the second optical fiber 29 (refer to FIG. 16) incorporated in the endoscope inserting portion 14; and a fifth connector 16e which receives and transmits a signal for controlling the scanning mirror 140 in the confocal optical system 137.

A description is given of a connecting relationship among the connectors 16a, 16b, 16d, and 16e. The first connector 16a is detachably connected to the socket 3a in the light source device 3. The second connector 16b is detachably connected to the socket 2a in the video processor 2 via a curling cord. Further, the fourth connector 16d is optically connected to the optical unit 7. The fifth connector 16e in the connector portion 16 is detachably connected to the scanning control device 6.

Laser beams are generated by the confocal observation LD 26 and are inputted to the fourth connector 16d arranged to the connector portion 16 in the endoscope 1 via the connector 7a and the optical fiber 28 from the first end portion 25a in the four-terminal coupler 25. The laser beams pass through the second optical fiber 29 arranged to the endoscope edge-portion 18 from the operating portion 15 in the endoscope 1, which is connected via the connecting cord 17. Further, the laser beams are transmitted to the confocal optical system 137 arranged to the endoscope edge-portion 18.

The operating portion 15 in the endoscope 1 has, at a predetermined position, operating means on the hand side such as the bending knob 32 for bending the bending portion 19 in an arbitrary direction.

Next, a description is given of the internal structure of the endoscope edge-portion 18.

Referring to FIG. 16, the endoscope edge-portion 18 has, at predetermined positions, the normal observation optical system 136, the confocal optical system 137, a light guide bundle 71 for illumination as illuminating means, and a treatment tool channel (not shown).

In the endoscope observing window 23 which is opened at the edge surface of the endoscope edge-portion 18, a wavelength split element 139 is arranged. The wavelength split element 139 has a wavelength split surface 139a through which a visible light area passes and to which an infrared light area is reflected. The normal observation optical system 136 is arranged on an optical path of the wavelength split surface 139a in a transmitting direction. The confocal optical system 137 is arranged on an optical path in the reflecting direction. The wavelength split element 139 is flat on the-subject side and a concave lens surface 139b is formed onto an opposed surface of the normal observation optical system 136.

The normal observation optical system 136 has a plurality of lens groups 136a arranged on the optical path and a solid image pick-up device 136b which is arranged in opposite to an image forming position of the lens groups 136a.

The confocal optical system 137 comprises: a first convex lens 137a which is arranged in opposite to the reflecting direction of the wavelength split surface 139a; a first fixing mirror 137b which is close to the first convex lens 137a and which is arranged in opposite to the optical axis of the first convex lens 137a at an inclination angle of 145°; a second convex lens 137c which is arranged near the first fixing mirror 137b; the scanning mirror 140 which is arranged near the second convex lens 137c; a second fixing mirror 137d which is arranged in parallel with the surface of the scanning mirror 140; and a third convex lens 137e which is arranged in opposite to the reflecting direction of the second fixing mirror 137d. The third convex lens 137e is arranged in opposite to an end portion of the second optical fiber 29.

In the confocal optical system 137 according to the sixth embodiment, the optical path is bent through four-time reflection by the wavelength split surface 139a, the first fixing mirror 137b, the scanning mirror 140, and the second fixing mirror 137d.

Next, the structure of the scanning mirror 140 will be described with reference to FIG. 17. A flat-formed gimbal ring 141 is arranged in an outer frame 140a in the scanning mirror 140. The center portion on the top and in the bottom of the gimbal ring 141 is supported to the inner periphery of the outer frame 140a via an outer hinge 142. An opening portion 141a is inserted in the center of the gimbal ring 141, and a first movable mirror 143 is arranged to the opening portion 141a. As shown in FIG. 17, the center portion on the right and left sides of the first movable mirror 143 is supported via an inner hinge 144 to the inner periphery of the gimbal ring 141.

The outer hinge 142 and the inner hinge 144 can be modified by twist. The modification due to the twist of the outer hinge 142 rotates and displaces the gimbal ring 141 with the outer hinge 142 as center. The modification due to the twist of the inner hinge 144 rotates and displaces the first movable mirror 143 with the inner hinge 144 as center.

Electrodes 145a and 145b (hatched in FIG. 17) are arranged to the left and right surfaces sandwiching the outer hinge 142 on the gimbal ring 141. Electrodes 146a and 146b (hatched in FIG. 17) are arranged to the top and bottom surfaces sandwiching the inner hinge 144 on the rear surface of the first movable mirror 143. The electrodes 145a and 145b and the electrodes 146a and 146b are connected to the scanning control device 6 via a wiring 147 (refer to FIG. 16). The scanning control device 6 controls voltages applied to the electrodes 145a and 145b and the electrodes 146a and 146b, thereby displacing the gimbal ring 141 and the first movable mirror 143 with static absorbing force between counter-electrodes (not shown) as driving force. Further, the infrared light reflected from the first movable mirror 143 is deviated in the two-dimensional direction (X-axis and Y-axis directions) perpendicular to the optical axis (Z-axis direction).

Next, the operation with the structure will be described according to the sixth embodiment.

In the normal observation using the visible light as the light source, the visible light outputted from the light source device 3 is inputted in the connector portion 16 via the first connector 16a, and is inputted to an input end of the light guide bundle 71 for illumination which is optically connected in the connector portion 16. The output end side of the light guide bundle 71 for illumination is inserted in the endoscope 1, the visible light is guided to the illuminating window 21 for endoscope observation which is opened to the edge surface of the endoscope edge-portion 18 via the light guide bundle 71 for illumination, and the visible light is illuminated to the subject from the illuminating window 21 for endoscope observation.

The visible light reflected from the subject is inputted to the wavelength split element 139 which is arranged to the endoscope observing window 23 opened to the edge surface of the endoscope edge-portion 18, passes through the wavelength split surface 139a, and further passes through the lens groups 136a in the normal observation optical system 136. And, the subject image is formed on the image pick-up surface of the solid image pick-up device 136b.

The subject image formed on the image pick-up surface is converted into an electrical signal and is transmitted to the video processor 2. The video processor 2 converts the electrical signal to a video signal by an incorporated processing circuit (not shown), and the video signal is outputted to the endoscope observation monitor 5. The subject image is displayed onto the endoscope observation monitor 5.

In the normal observation, when the concerned portion such as the lesion portion is found, the confocal observation starts.

In the confocal observation, the scanning control device 6 applies a control voltage to the respective electrodes 145a, 145b and 146a, 146b arranged to the scanning mirror 140. The infrared light reflected from the reflecting surface of the first movable mirror 143 is deviated in the two-dimensional direction (X-axis and Y-axis directions) perpendicular to the optical axis (Z-axis direction) by the control voltage. Then, the first movable mirror 143 starts to be driven.

Next, the confocal observation LD 26 arranged to the optical unit 7 starts. Then, the infrared light from the confocal observation LD 26 is outputted from the end portion of the second optical fiber 29 arranged to the endoscope edge-portion 18 via the second optical fiber 29.

The infrared light outputted from the end portion of the second optical fiber 29 is inputted to the wavelength split element 139 via the third convex lens 137e and the second fixing mirror 137d in the confocal optical system 137 arranged to the end portion thereof, the first movable mirror 143 arranged to the scanning mirror 140 (refer to FIG. 17), the second convex lens 137c, the first fixing mirror 137b, and the first convex lens 137a. The infrared light is reflected by the wavelength split surface 139a in the wavelength split element 139. The optical path of the wavelength split element 139 is bent at an angle of 90°, and is illuminated to the subject from the endoscope observing window 23 opened to the edge surface of the endoscope edge-portion 18.

Then, the displacement of the first movable mirror 143 arranged to the scanning mirror 140 enables the infrared light to be displaced in the two-dimensional direction (X-axis and Y-axis directions). Therefore, the infrared light scans the subject two-dimensionally. The infrared light reflected and scattered by the subject is inputted to the wavelength split element 139 from the endoscope observing window 23 opened to the edge surface of the endoscope edge-portion 18. Then, the infrared light is reflected by the wavelength split surface 139a in the wavelength split element 139, and is inputted to the first convex lens 137a forming the confocal optical system 137. The infrared light is inputted from the first convex lens 137a to the second optical fiber 29 via an optical path inverse to the advancing optical path.

The infrared light inputted to the second optical fiber 29 passes through the second optical fiber 29, is inputted to the PMT unit 27 in the optical unit 7, and is converted into an electrical signal. The electrical signal photoelectrically converted by the PMT unit 27 is transmitted to the filter device 8 via the signal line 8a connected via the connector 7b to an output end of the PMT unit 27. Further, the electrical signal passing though the filter device 8 is inputted to the imaging device 9 and is imaged. After that, the image is outputted to the confocal image monitor 10′ and a confocal image of the subject is displayed on the confocal image monitor 10′.

According to the sixth embodiment, the wavelength split element 139 is arranged to the endoscope observing window 23 opened to the edge surface of the endoscope edge-portion 18, namely, at the position closest to the subject side of the endoscope 1. The wavelength split surface 139a arranged to the wavelength split element 139 splits the visible light and the infrared light into the normal observation optical system 136 and the confocal optical system 137. Thus, the lens positioned at the position closest to the subject side of the normal observation optical system 136 becomes a concave lens and the wide-viewing observation is possible in the normal observation using the endoscope.

Since the convex lens is the lens at the position closest to the subject side of the confocal optical system 137, the confocal observation is possible with the high number of aperture (NA).

(Seventh Embodiment)

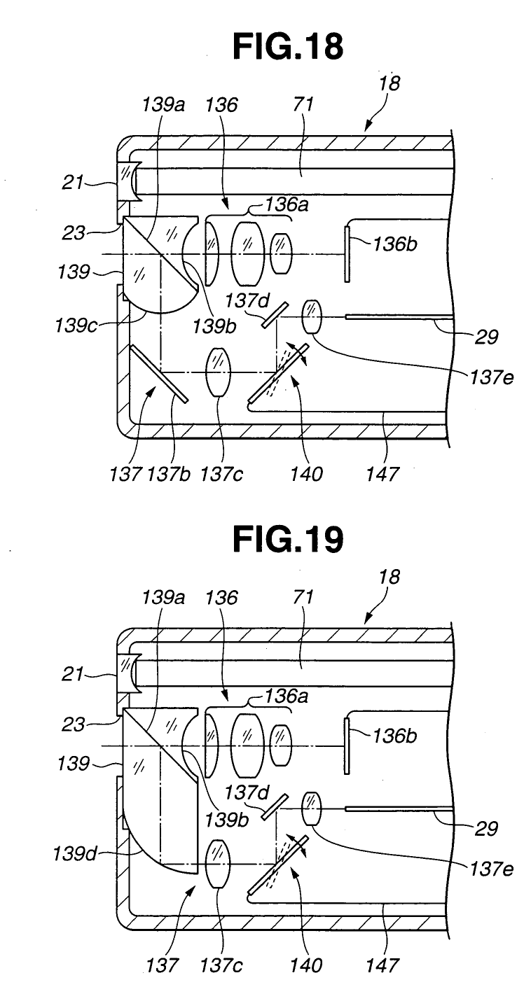

FIG. 18 shows a cross-sectional view of the endoscope edge portion according to the seventh embodiment.

According to the seventh embodiment, an opposed surface of the confocal optical system 137 in the wavelength split element 139 is set as a convex lens surface 139c having the same function as that of the first convex lens 137a in the confocal optical system 137 arranged according to the sixth embodiment.

The confocal optical system 137 integrally includes the convex lens surface 139c having the same function as that of the first convex lens 137a, thus to omit the first convex lens 137a shown according to the sixth embodiment, and the space is saved. Other operation and advantage are the same as those according to the sixth embodiment and therefore a description thereof is omitted.

(Eighth Embodiment)

FIG. 19 shows a cross-sectional view of the endoscope edge portion according to the eighth embodiment.

According to the eighth embodiment, a surface opposed to the confocal optical system 137 of the wavelength split element 139 is as a concave mirror 139d having the same function as those of the first convex lens 137a and the first fixing mirror 137b in the confocal optical system 137 arranged according to the sixth embodiment.

The confocal optical system 137 integrally includes the concave mirror 139d having the same functions as those of the first convex lens 137a and the first fixing mirror 137b. Consequently, as compared with that according to the seventh embodiment, the space is further saved. Other operation and advantage are the same as those according to the sixth embodiment and, therefore, a description thereof is omitted.

(Ninth Embodiment)

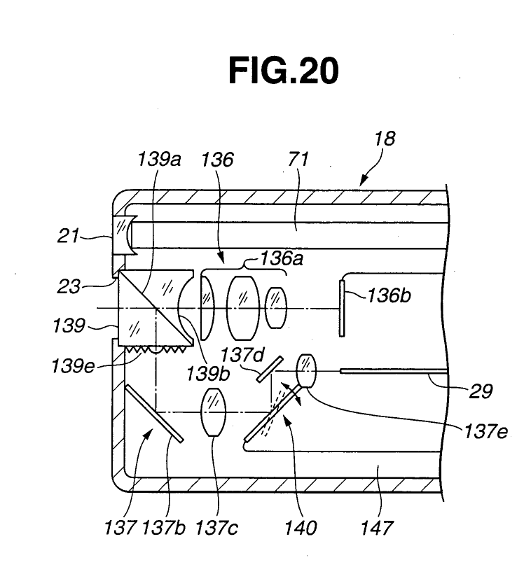

FIG. 20 shows a cross-sectional view of the endoscope edge portion according to the ninth embodiment.

According to the ninth embodiment, a surface opposed to the confocal optical system 137 of the wavelength split element 139 integrally includes a diffraction-type optical device surface 139e having the same function as that of the first convex lens 137a in the confocal optical system 137 arranged according to the sixth embodiment.

The confocal optical system 137 integrally includes the diffraction-type optical device surface 139e having the same function as that of the first convex lens 137a. Consequently, the first convex lens 137a according to the sixth embodiment is omitted, in accordance therewith, the number of parts of the confocal optical system 137 is reduced and the space is saved.

Further, a diffraction pattern of the diffraction-type optical device surface 139e does not need to have a processed complicated curve and therefore the manufacturing is simple and the manufacturing costs are reduced because the diffraction pattern is formed by using a molding arranged on the plain. Other operation and advantage are the same as those according to the sixth embodiment and, therefore, a description thereof is omitted.

(Tenth Embodiment)

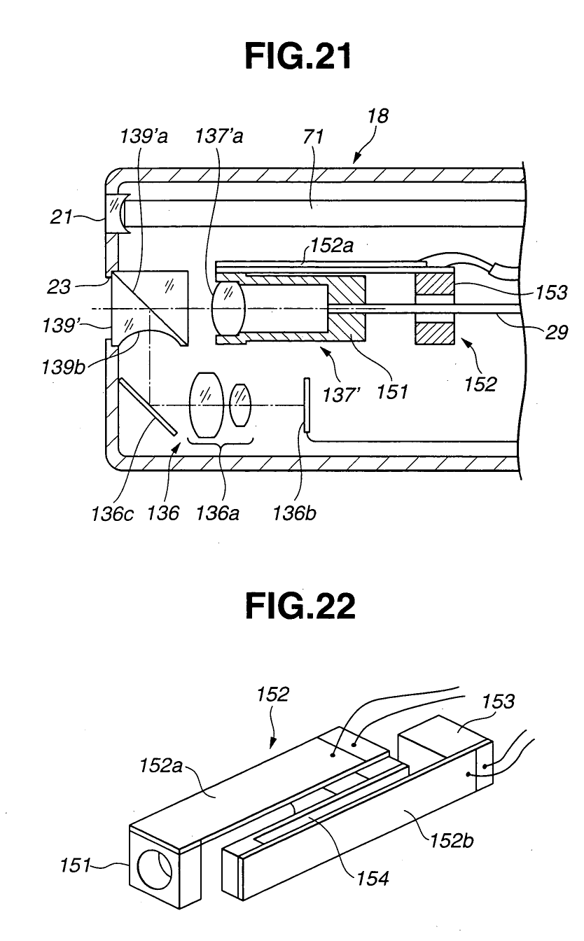

FIGS. 21 and 22 show the tenth embodiment of the present invention. FIG. 21 is a cross-sectional view showing the endoscope edge portion. FIG. 22 is a perspective view showing a piezoelectric actuator.

The wavelength split surface 139a arranged to the wavelength split element 139 according to the first to ninth embodiments has the property for transmitting the visible light area and reflecting the infrared light area. However, according to the tenth embodiment, a wavelength split element 139′a in a wavelength split element 139′ has the property for reelecting the visible light area and transmitting the infrared light area.

A confocal optical system 137′ is arranged in the optical path in the transmitting direction of the wavelength split surface 139′a arranged to the wavelength split element 139′. The normal observation optical system 136 is arranged to the optical path in the reflecting direction of the wavelength split element 139′.

Specifically, a surface on the subject side of the wavelength split element 139′ and an opposed surface thereof, namely, surfaces in the transmitting direction are flat-shaped. The concave lens surface 139b is formed in the reflecting direction of the wavelength split element 139′a as a surface with a right angle to the subject side.

The normal observation optical system 136 has a third fixing mirror 136c oppositely arranged to the optical axis from the wavelength split element 139′ at an inclination angle of 145°. Further, the lens groups 136a and the solid image pick-up device 136b are arranged in the reflecting direction of the third fixing mirror 136c.

The confocal optical system 137′ arranged in the transmitting direction of the wavelength split element 139′ has the first convex lens 137′a arranged adjacently to the rear end surface of the wavelength split element 139′. An end surface of the second optical fiber 29 is oppositely arranged to the first convex lens 137′a. Similarly to the sixth embodiment, the second optical fiber 29 is optically connected to the fourth connector 16d arranged to the connector portion 16 (refer to FIG. 15). The first convex lens 137′a and the end portion of the second optical fiber 29 are fixed to the mirror frame 151, and the mirror frame 151 is continuously arranged to a flat piezoelectric actuator unit 152.

The flat piezoelectric actuator unit 152 displaces the edge side of the mirror frame 151 in the two-dimensional direction (X-axis and Y-axis directions) perpendicular to the optical axis (Z-axis direction) of the mirror frame 151, and two-dimensionally scans the subject with the infrared light outputted from the second optical fiber 29. Referring to FIG. 22, the flat piezoelectric actuator unit 152 has a first flat piezoelectric actuator 152a which displaces the mirror frame 151 in the vertical direction (Y-axis direction) and a second flat piezoelectric actuator 152b which displaces the mirror frame 151 in the horizontal direction (X-axis direction).

Th rear end portion of the second flat piezoelectric actuator 152b is fixed to a base 153 fixed to the endoscope edge-portion 18. The front end portion of the second flat piezoelectric actuator 152b is fixed to the front end portion of a connecting member 154. The connecting member 154 is substantially Z-shaped, and the rear end of the first flat piezoelectric actuator 152a is fixed to the rear end portion of the connecting member 154. Further, the mirror frame 151 is fixed to the edge side of the first flat piezoelectric actuator 152a.

The first flat piezoelectric actuator 152a and the second flat piezoelectric actuator 152b are cantilevers which are formed by adhering, to a thin metallic plate, flat piezoelectric elements that are subjected to polarization in the thickness direction of the piezoelectric plates. A voltage is applied to the first flat piezoelectric actuator 152a and the second flat piezoelectric actuator 152b in the same direction as that of the polarization of the piezoelectric plates, thereby causing the displacement so as to make the thickness of the piezoelectric plates thicker and to reduce the length of the piezoelectric plates in the longitudinal direction. In this case, since the flat piezoelectric elements are adhered to the metallic plate, the bending displacement is caused so that the piezoelectric elements are bent inside like arc. A voltage is applied in the direction inverse to the polarization of the piezoelectric plates, and then the displacement is caused so as to make the thickness of the piezoelectric plates thinner and to increase the length of the piezoelectric plates in the longitudinal direction. In this case, the bending displacement is caused so that the piezoelectric elements are bent outside like arc.

As a result of the displacement of the piezoelectric elements, the mirror frame 151 and the connecting member 154 fixed to free end sides of the first flat piezoelectric actuator 152a and the second flat piezoelectric actuator 152b are displaced in the same direction. The edge side of the mirror frame 151 is displaced in the two-dimensional directions (X-axis and Y-axis directions) perpendicular to the optical axis (Z-axis direction), and the subject is two-dimensionally scanned with the infrared light outputted from the second optical fiber 29.

The amount of displacement of the first flat piezoelectric actuator 152a and the second flat piezoelectric actuator 152b on the free end sides are controlled by applying, to the piezoelectric elements, control voltages outputted from the scanning control device 6 (refer to FIG. 15). According to the tenth embodiment, the raster-scanning is performed with the main scanning of one of the first flat piezoelectric actuator 152a and the second flat piezoelectric actuator 152b and with the sub-scanning of another.

With the above-mentioned structure, in the normal observation using the visible light as the light source, the visible light outputted from the light source device 3 (refer to FIG. 15) is illuminated from the illuminating window 21 for endoscope observation opened to the edge surface of the endoscope edge-portion 18 via the light guide bundle 71 for illumination, and further illuminates the field of view of the normal observation optical system 136.

The illuminated visible light is reflected and is scattered by the subject, and is inputted to the wavelength split element 139. The visible light is further reflected by the wavelength split surface 139a arranged to the wavelength split element 139, is bent at an angle of 90°, and passes through the normal observation optical system 136 comprising the lens groups 136a and the third fixing mirror 136c. The subject image is formed onto the image pick-up surface of the solid image pick-up device 136b.

The subject image formed onto the image pick-up surface of the solid image pick-up device 136b is converted into an electrical signal, and is transmitted to the video processor 2 (refer to FIG. 15). The video processor 2 converts the electrical signal into a video signal by an incorporated processing circuit (not shown), and the video signal is outputted to the endoscope observation monitor 5. The subject image is displayed on the endoscope observation monitor 5.

In the normal observation, when the concerned portion such as the lesion portion is found, the confocal observation starts. In the confocal observation, control voltages are applied from the scanning control device 6 to the first flat piezoelectric actuator 152a and the second flat piezoelectric actuator 152b arranged in the flat piezoelectric actuator unit 152, and the driving of the first flat piezoelectric actuator 152a and the second flat piezoelectric actuator 152b starts.

Next, the confocal observation LD 26 (refer to FIG. 15) arranged to the optical unit 7 starts. Then, the infrared light from the confocal observation LD 26 passes through the second optical fiber 29. The subject is illuminated with the infrared light from the end portion of the second optical fiber 29 in the endoscope edge-portion 18 via the first convex lens 137′a and the wavelength split element 139′.

In this case, since the mirror frame 151 two-dimensionally raster-scans the subject by the driving of the flat piezoelectric actuator unit 152, the subject is two-dimensionally raster-scanned with the infrared light.

The infrared light reflected and scattered on the subject is inputted to the wavelength split element 139′ again, is transmitted through the wavelength split surface 139a, passes through the first concave lens 137′a, and is inputted to the second optical fiber 29. The infrared light inputted to the second optical fiber 29 passes through the second optical fiber 29, is inputted to the PMT unit 27 arranged to the optical unit 7, and is converted into the electrical signal. The electrical signal photoelectrically converted by the PMT unit 27 passes through the signal line 8a connected to the output terminal of the PMT unit 27 via the connector 7b, and is transmitted to the filter device 8.

Further, the electrical signal passing though the filter device 8 is inputted to the imaging device 9, and is imaged. Then, the signal is outputted to the confocal image monitor 10′, and the confocal image of the subject is displayed on the confocal image monitor 10′.

According to the tenth embodiment, the subject is two-dimensionally scanned by the mirror frame 151 for holding the second optical fiber 29 which is driven by the flat piezoelectric actuator unit 152. Therefore, as compared with the case of scanning the subject by using the scanning mirror 140 according to the first to ninth embodiments, the number of bending times of the optical path is reduced and, in accordance therewith, the structure is simplified.

(Eleventh Embodiment)

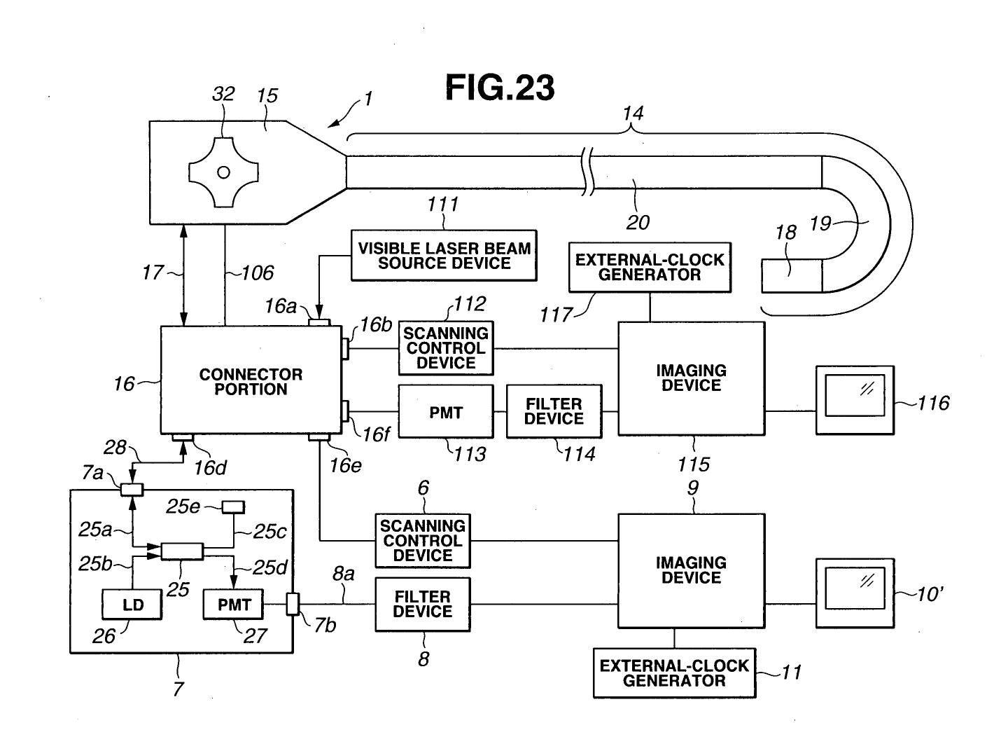

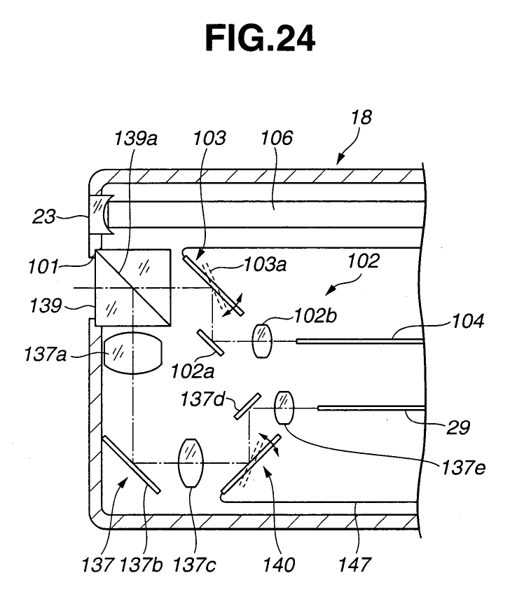

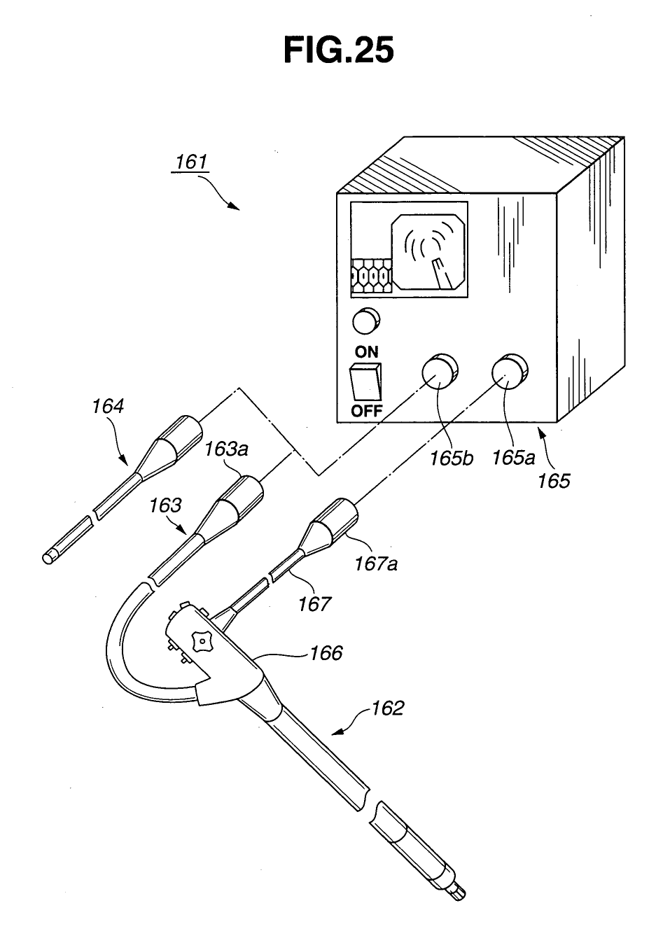

FIGS. 23 and 24 show the eleventh embodiment of the present invention. FIG. 23 is a diagram showing the structure of an endoscope. FIG. 24 is a cross-sectional view showing an endoscope edge portion.

According to the sixth embodiment, the light source in the normal observation uses the simple visible light. However, according to the eleventh embodiment, the light source uses visible laser beams and the normal observation is performed by using the visible laser beams.

Referring to FIG. 23, an endoscope apparatus includes a normal observing system using the visible laser beams as the light source, in place of the normal observing system using the simple visible light as the light source according to the sixth embodiment. The confocal observing system using the infrared light has the same structure as that according to the sixth embodiment. Therefore, the same components are designated by the same reference numerals and a description thereof is omitted here.

First, a description is given of the internal structure of the endoscope edge-portion 18. Referring to FIG. 24, an illumination observing window 101 and the endoscope observing window 23 are opened to the edge surface of the endoscope edge-portion 18. The wavelength split element 139 is attached to the illumination observing window 101. The wavelength split surface 139a in the wavelength split element 139 has the property for transmitting the visible light area and reflecting the infrared light area. Therefore, the confocal optical system 137 is arranged onto the optical path in the reflecting direction of the wavelength split surface 139a, similarly to the sixth embodiment.

A scanning optical system 102 for illumination is arranged onto the optical path in the transmitting direction of the wavelength split surface 139a. The scanning optical system 102 for illumination comprises a scanning mirror 103 for illumination which is arranged to the rear end surface of the wavelength split element 139, a fourth fixing mirror 102a which is oppositely arranged in the reflecting direction of the scanning mirror 103 for illumination, and a fourth convex lens 102b which is oppositely arranged in the reflecting direction of the fourth fixing mirror 102a. Further, an output end surface of the optical fiber 104 for illumination is oppositely arranged to the fourth convex lens 102b, as illuminating means for illumination with the visible laser beams.

The scanning mirror 103 for illumination has the same structure as that of the scanning mirror 140 (refer to FIG. 17) according to the sixth embodiment. In the scanning mirror 103 for illumination, a second movable mirror 103a shown by a broken line in FIG. 24 is displaced by a control voltage which is energized via the wiring 105 from the scanning control device 112, which will be described later. The subject is scanned with the visible laser beams reflected by the second movable mirror 103a in the two-dimensional direction (X-axis and Y-axis directions) to the optical axis (Z-axis direction). In this case, the second movable mirror 103a corresponds to the first movable mirror 143 shown in FIG. 17.

The endoscope observing window 23 has an input end surface of a light guide 106 which receives and transmits the scattering light or reflecting light from the subject illuminated by the visible laser beams. Referring to FIG. 23, the light guide 106 is inserted in the endoscope 1 and enter the connector portion 16. Further, the light guide 106 is optically connected to a tenth connector 16f in the connector portion 16.

In the connector portion 16, the first connector 16a is optically connected to the optical fiber 104 for illumination. The second connector 16b is electrically connected to the wiring 105 extending from the scanning control device 112. Similarly to the sixth embodiment, a wiring 147 extending from the second optical fiber 29 and the scanning mirror 140 is connected to the fourth connector 16d and the fifth connector 16e.

Referring to FIG. 23, similarly to the sixth embodiment, the optical unit 7 and the scanning control device 6 are connected to the fourth connector 16d and the fifth connector 16e in the connector portion 16. A visible laser-beam source device 111 for generating the visible laser beams is optically connected to the first connector 16a. A scanning control device 112 for driving the scanning mirror 103 for illumination is electrically connected to the second connector 16b. Further, a PMT unit 113 for receiving the light from the light guide 106 and converting it into the electrical signal is connected to the tenth connector 16f.

A filter device 114 for passing only a specific frequency component from the PMT unit 113 is connected to the PMT unit 113. The filter device 114 is connected to an imaging device 115 which images the electrical signal from the filter device 114. Further, the imaging device 115 is connected to a normal observing monitor 116 which displays the video signal from the imaging device 115. An external-clock generator 117 for generating a clock pulse as a reference of a driving waveform for driving the scanning mirror 103 for illumination is connected to the imaging device 115.

With above structure, in the normal observation, the visible laser beams generated from the visible laser beam source device 111 are outputted from the scanning optical system 102 for illumination arranged in the endoscope edge-portion 18 via the optical fiber 104 for illumination, passing through the wavelength split element 139, and the subject is illuminated by the raster scanning with the visible laser beams.

The visible laser beams illuminated the subject are reflected and scattered by the subject, and are inputted to the light guide 106. The visible laser beams inputted to the light guide 106 pass through the light guide 106, are inputted to the PMT unit 113, and are converted into an electrical signal.

The electrical signal photoelectrically converted by the PMT unit 113 is transmitted to the imaging device 115 via the filter device 114. In the imaging device 115, the electrical signal is converted into a video signal by a processing circuit (not shown). The signal is outputted to the normal observing monitor 116, and the subject image is displayed on the normal observing monitor 116.

In the normal observation, when the concerned portion such as the lesion portion is found, the confocal observation starts. Since the confocal observation is the same as that according to the sixth embodiment, a description thereof is omitted here.

According to the eleventh embodiment, the optical path of the scanning optical system 102 for illumination for guiding the visible laser beams for the normal observation and the optical path of the confocal optical system 137 for guiding the infrared light for the confocal observation are shared in the wavelength split element 139 arranged to the endoscope edge-portion 18 closest to the subject. Thus, the endoscope edge-portion 18 is made thinner.

Having described the preferred embodiments of the invention referring to the accompanying drawings, it should be understood that the present invention is not limited to the those precise embodiments and various changes and modifications thereof could be made by one skilled in the art without departing from the spirit or scope of the invention as defined in the appended claims.

What is claimed is:

1. An endoscope comprising:
an inserting portion which can be inserted in a specimen;
an optical system for endoscope observation arranged in an edge of the inserting portion;
an optical system for microscopic observation which is arranged in the edge of the inserting portion and which microscopically observes an observing portion of the specimen;
a moving mechanism for adjusting an angle of view which is arranged in the edge of the inserting portion and which changes the angle of view by moving a part of the optical system for endoscope observation; and
a focal-point adjusting mechanism which is arranged in the edge of the inserting portion and which moves a focal point on the side of a subject in the optical system for microscopic observation,
wherein the moving mechanism for adjusting the angle of view and the focal-point adjusting mechanism are arranged forward and backward of the same axis to the inserting portion.

2. An endoscope according to claim 1, wherein the moving mechanism for adjusting the angle of view and the focal-point adjusting mechanism are arranged independently.

3. An endoscope according to claim 1, wherein the moving mechanism for adjusting the angle of view and the focal-point adjusting mechanism are arranged integrally.

4. An endoscope according to claim 1, wherein the moving mechanism for adjusting the angle of view and the focal-point adjusting mechanism have an actuator using a piezoelectric element.

5. An endoscope according to claim 1, wherein the optical system for microscopic observation is projected in front of the optical system for endoscope observation, by the same distance as that between the observing portion and an optical-system edge lens when the angle of view of the optical system for endoscope observation is minimum.

6. An endoscope according to claim 3, wherein an elastic member intervenes between the moving mechanism for adjusting the angle of view and the optical system for microscopic observation so that the moving mechanism for adjusting the angle of view can be moved out of a focal-point adjusting range of the optical system for microscopic observation.

7. An endoscope according to claim 1, wherein a heating portion in the moving mechanism for adjusting the angle of view is arranged to a portion other than the edge portion of the inserting portion, and
only a mechanism for changing an operating direction is arranged to the edge portion of the inserting portion.

8. An endoscope according to claim 1, wherein the optical system for microscopic observation is a confocal optical system.

9. An endoscope according to claim 1, wherein the optical system for microscopic observation comprises:
a high-magnification enlarging optical system;
image pick-up means;
a transparent optical window which is arranged forward of the high-magnification enlarging optical system and which can be touched to the subject; and
illuminating means which is independent of illuminating means for endoscope observation, and
an observing range of the high-magnification enlarging optical system is approximately 0 to 100 μm from the optical window.

10. An endoscope comprising:
an inserting portion which can be inserted in a specimen;
illuminating means which is arranged in an edge of the inserting portion;
a normal observing optical system which is arranged in the edge of the inserting portion and which forms an image of an illuminated subject; and
a confocal optical system which is arranged in the edge of the inserting portion with a high resolution, and
a waveform spilt element which is shared by the normal observing optical system and the confocal optical system is arranged to the closest side of the subject.

11. An endoscope according to claim 10, wherein a lens surface is integrally formed to the waveform split element.

12. An endoscope according to claim 11, wherein the lens surface is a concave lens surface which is formed on the side of the normal observing optical system.

13. An endoscope according to claim 11, wherein the lens surface is a convex lens surface which is formed on the side of the confocal optical system.

14. An endoscope according to claim 11, wherein the lens surface is a concave mirror which is formed on the side of the confocal optical system.

15. An endoscope according to claim 11, wherein the lens surface comprises a diffraction-type optical device.

16. An endoscope according to claim 10, wherein the illuminating means is a light guide bundle, and the normal observation optical system is an optical system which forms a two-dimensional image on a solid image pick-up device.

17. An endoscope comprising:
an inserting portion which can be inserted in a specimen;
a scanning optical system for illumination which is arranged in an edge of the inserting portion;
a light guide which is arranged in the edge of the inserting portion and which receives and transmits scattering light or reflecting light from a subject illuminated by the scanning optical system for illumination; and
a confocal optical system which is arranged in the edge of the inserting portion with a high resolution,
wherein the scanning optical system for illumination and the confocal optical system share a common wavelength split element at the position closest to the subject side.

18. An endoscope according to claim 17, wherein the confocal optical system is a scanning optical system comprising a scanning mirror for two-dimensional deviation.

19. An endoscope according to claim 17, wherein the confocal optical system is a scanning optical system including a structure in which two piezoelectric actuators driven in each single direction are arranged so as to make the displacing directions thereof perpendicular to each other.
